(19) United States
(12) Patent Application Publication
(10) Pub. No.: US 2017/0319106 A1
(43) Pub. Date: Nov. 9, 2017

(54) MEASUREMENT OF RESPIRATORY FUNCTION

(71) Applicant: Digidoc Technologies AS, Egersund (NO)

(72) Inventors: Damoun Nassehi, Egersund (NO); Carl Andre Norstebo, Oslo (NO); Jakob Nygren Ostergard, Egersund (NO); Johan Schreiner, Oslo (NO)

(21) Appl. No.: 15/528,130

(22) PCT Filed: Nov. 20, 2015
(86) PCT No.: PCT/EP2015/077295

§ 371 Date: May 19, 2017

(30) Foreign Application Priority Data
Nov. 20, 2014 (GB) 1420669.2
Aug. 26, 2015 (GB) 1515172.3

(51) Int. Cl. A61B 5/087; A61B 5/00; G10K 5/00

(52) U.S. Cl. CPC A61B 5/0871; A61B 5/7278; A61B 5/087; G10K 5/00

(57) ABSTRACT

The present invention provides a method of measuring respiratory flow rate. The method includes a user exhaling through a flow dependent sound producing device which comprises a mouthpiece and a vortex chamber. The vortex chamber has an axis and an outlet such that exhaled air flows through the mouthpiece into the vortex chamber causing the exhaled air to form a vortex around the axis and then pass out of the chamber via the outlet in an axial direction, thereby producing a sound. The method further includes detecting the sound using a mobile electronic device and analyzing said sound to determine a frequency of said sound and using said frequency to determine the respiratory flow rate.

This application is entitled to the benefit of, and incorporates by reference essential subject matter disclosed in PCT Application No. PCT/EP2015/077295 filed on Nov. 20, 2015, which claims priority to Great Britain Application No. 1420669.2 filed Nov. 20, 2014 and Great Britain Application No. 1515172.3 filed Aug. 26, 2015.

### BACKGROUND OF THE INVENTION

### 1. Technical Field

This invention relates to the measurement of human respiratory flow rate.

### 2. Background Information

Measuring a person's maximum expiratory flow rate can be useful to determine increased airway resistance that is indicative of many respiratory issues such as asthma, chronic bronchitis and emphysema (or other types of chronic obstructive pulmonary disease).

Until recently, measuring respiratory flow rates has generally only been possible at a hospital or clinic using dedicated, sometimes expensive, equipment typically using a flow meter which measures the flow rate of air exhaled by a user. The peak expiratory flow rate (PEFR) or “peak flow” is the maximum flow rate of the exhaled air. This must then be interpreted by a clinician to determine whether it is satisfactory or a cause for concern and possible treatment.

Previously it has been proposed in “Measurement of peak expiratory flow rate with a whistle”—British Medical Journal 30 Oct. 1965, page 1040-1041, to measure flow rate in a mobile manner through the use of a plastic tube with a slot in the side and double-orifice member at the end wherein a slot in the side of the tube acts as a leak hole. This device is operated by blowing into it and moving the plastic tube until an audible sound is no longer produced. By sliding the plastic tube to the point in which sound is no longer produced it is possible to read a scale on the side of the tube which indicates the user's respiratory flow rate. This method is not very accurate however and typically yields readings within a wide range of values.

### SUMMARY OF THE INVENTION

When viewed from a first aspect the present invention provides a method of measuring respiratory flow rate comprising: a user exhaling through a flow dependent sound producing device which comprises a mouthpiece and a vortex chamber having an axis and an outlet such that exhaled air flows through the mouthpiece into the vortex chamber causing the exhaled air to form a vortex around the axis and then pass out of the chamber via the outlet in an axial direction, thereby producing a sound; detecting the sound using an electronic device; and analyzing said sound to determine a frequency of said sound and using said frequency to determine the respiratory flow rate.

When viewed from a second aspect the present invention provides an apparatus for measuring respiratory flow rate comprising: a flow dependent sound producing device comprising a mouthpiece and a vortex chamber having an axis and an outlet such that in use exhaled air flowing through the mouthpiece into the vortex chamber forms a vortex around the axis and passes out of the chamber via the outlet in an axial direction, thereby producing a sound; an electronic device capable of detecting the sound; a sound analysis module configured to determine a frequency of the sound; and a processor configured to determine the respiratory flow rate from said frequency.

Thus it can be seen by those skilled in the art that in accordance with the invention respiratory flow rate can be measured electronically using a very simple accessory that does not require moving parts, and an electronic device such as a smartphone, tablet computer, smart watch or other wearable device, personal digital assistant (PDA), mobile gaming system etc. Such an arrangement is easy to use and inexpensive to produce—e.g. it is possible in preferred embodiments for the accessory (i.e. flow dependent sound producing device or “vortex whistle”) to be manufactured from two injection molded plastic components, however it may be possible to manufacture some embodiments from more components or a single component. This reduction in cost could make it more readily available for the general public.

The advantage of using the flow dependent sound producing device set out is that it has been found to have a linear relationship between the volume air flow rate and the frequency of the sound produced over a wide range and thus it can be easily calibrated for both adults and children. In other words, the flow dependent sound producing device (or “vortex whistle”) provides a substantially linear relationship between the respiratory flow rate and the frequency of the sound produced. This linear relationship distinguishes the flow dependent sound producing device or “vortex whistle” from other spirometers e.g. using a fluidic oscillator. For example a typical adult flow rate is 400 liters/minute (lpm) whereas children typically have flow rates of the order of 150 liters/minute (lpm). This means that only one component needs to be tooled for all applications. It will be appreciated that the frequency of the sound produced by air passing out of the vortex chamber varies with the flow rate of the exhaled air and hence the frequency will change if the flow rate changes, e.g. due to a reduction in flow rate during an exhalation event. The flow dependent sound producing device or “vortex whistle” also has the advantage of no moving parts. This can be contrasted with spirometric flow rate devices that require a rotor (e.g. a mechanical siren).

Preferably the electronic device is a mobile device. The mobile device is preferably a mobile digital device, optionally selected from a (non-exhaustive) list comprising one or more of: a smartphone, tablet computer, smart watch or other wearable device, personal digital assistant (PDA), or mobile gaming system. The use of a mobile device and simple accessory enables the user to take measurement of their respiratory flow rate where and when they desire. This opens up the possibility of a user being able to make an association between specific reasons, circumstances and reductions in their respiratory flow rate. For example, reductions may be related to specific times of day or year or specific places or environments. This is particularly advantageous as it may be that such specific circumstances that cause abnomtalities in the flow rate would not be noticed if the tests were consistently carried out in a clinic away from problem environments.

In some instances the flow dependent sound producing device may be able to produce a relatively loud sound e.g. when a user with a strong flow rate exhales into the device. This may be a particular problem for adult users rather than children. The Applicant appreciates that this can be problematic as, when the sound produced by the flow dependent sound producing device is too loud, it can saturate or distort the electronic device that is detecting the sound. This problem may be especially acute when the electronic device is a mobile device, because the separation distance between the two devices is then under the user's control and a user may hold the electronic device very close to the outlet of the sound producing device. Also the separation distance may not match the range over which the mobile device is optimized for sound detection. This is likely to result in inaccurate measurements of the sound and thus introduces inaccuracies in the calculation of the respiratory flow rate. Therefore, in a preferable set of embodiments the flow dependent sound producing device further comprises means for reducing the amplitude of the produced sound.

This is considered novel and inventive in its own right. Thus according to a further aspect of the present invention there is provided a flow dependent sound producing device comprising: a mouthpiece, for the intake of exhaled air, which is connected to a vortex chamber having an axis and an outlet such that in use exhaled air flowing through the mouthpiece into the vortex chamber forms a vortex around the axis and then passes out of the chamber via the outlet in an axial direction, thereby producing a sound, the flow dependent sound producing device further comprising means for reducing the amplitude of the produced sound.

By the introduction of means for reducing the amplitude of the sound the flow dependent sound producing device is able to produce a sound that does not saturate a detector or distort its measurement, e.g. when using a microphone of an electronic device to detect the sound. This may also make the device more suitable for home use, by reducing the amplitude of the produced sound so that it does not disturb a user.

In a set of embodiments the means for reducing the amplitude of the produced sound comprises a bypass air flow path allowing some of the exhaled air flowing through the mouthpiece to leave the flow dependent sound producing device without passing through the outlet of the vortex chamber. This is advantageous as it is the volume of air passing into the vortex chamber to form a vortex before exiting through the outlet which determines the amplitude of the sound produced. By reducing the volume of air passing out of the outlet it is possible to reduce the amplitude without affecting the relationship between air flow rate and the frequency of the sound produced. For example, at least some of the exhaled air entering the vortex chamber may be diverted to the bypass flow path rather than being entrained in the vortex and passing out via the outlet. In a preferred set of embodiments the bypass air flow path is arranged such that the air passing along the bypass air flow path does not pass into the vortex chamber. This is particularly advantageous as directly reducing the volume of air that enters the vortex chamber immediately reduces the amplitude of the produced sound without affecting the vortex.

In a preferred set of embodiments the flow dependent sound producing device comprises a secondary, bypass, outlet connected to the bypass air flow path. This allows the air passing along the bypass air flow path to escape the flow dependent sound producing device. It will be appreciated that the bypass outlet is independent of the vortex outlet.

In some embodiments the bypass air flow path is arranged such that air which passes through the bypass air flow path does not produce any sound. In some embodiments the bypass air flow path is arranged such that air which passes through the bypass air flow path does not produce an audible sound. In some embodiments the bypass air flow path is arranged such that air which passes through the bypass air flow path produces a secondary sound that is distinguishable from the primary sound produced by air exiting from the vortex chamber. For example, the secondary sound may have a different frequency spectrum to the primary sound. The Applicant has recognized that a secondary sound produced by the bypass air flow path may be exploited to assist in detection of the primary sound produced by the vortex chamber. An electronic device arranged to detect the primary sound may include a sound analysis module running noise cancellation algorithms and/or algorithms that recognize the primary sound. In a preferred set of embodiments the bypass air flow path is arranged to produce a secondary sound having a substantially constant frequency. It will be appreciated that such a secondary sound can be readily distinguished from the primary sound produced by the vortex whistle as the frequency of the primary sound has a linear relationship with air flow rate and therefore varies as the flow rate changes (e.g. falls) during exhalation. An electronic device may be arranged to detect the secondary sound as an indicator that a user is exhaling into the flow dependent sound producing device and use this indicator to actively detect the primary sound. For example, a smartphone app running on such an electronic device would know exactly when to “listen” for the primary sound produced by the vortex chamber. This is also advantageous as it may alleviate the need for a sound analysis module in an electronic device to include software that is programmed to recognize the primary sound.

In addition, or alternatively, the bypass air flow path may be arranged to produce a secondary sound having a frequency in a different range to the frequency of the primary sound. For example, the primary sound produced by the vortex chamber may have a frequency in the audible range of 20 Hz to 20 kHz. The bypass air flow path may be arranged to produce a secondary sound having a frequency outside this range, for example in the ultrasonic range of 20-100 kHz, 30-100 kHz, 40-100 kHz or 50-100 kHz. If the secondary sound is ultrasonic then it will not be heard by the user and only registered by the electronic device.

In a further preferred set of embodiments the bypass air flow path is constructed to be a substantially straight through flow path, for example extending externally around the vortex chamber, to avoid the generation of turbulence and thus avoid producing a sound. In various embodiments the bypass air flow path directs some of the exhaled air to pass out of the device in a straight-through direction via one or more bypass outlets. A straight-through direction will be understood to mean a direction in the general plane of the device, as compared to air exiting from the vortex chamber in an axial direction via the other outlet.

As mentioned above, the bypass air flow path may include the vortex chamber. For example, an additional outlet may be provided in the vortex chamber. Such an additional outlet in the vortex chamber may be arranged such that air which passes out through the additional outlet does not make a sound. In such arrangements some of the air in the vortex chamber is able to leave via the bypass air flow path rather than via the sound producing outlet. In a preferred set of embodiments, however, the bypass air flow path is arranged such that some of the exhaled air flowing through the mouthpiece enters the bypass air flow path before reaching the vortex chamber. This embodiment is particularly advantageous as it means that the vortex chamber can remain closed apart from a conduit extending from the mouthpiece and the outlet that allows air to pass out of the chamber in the axial direction. This helps to avoid any effects that providing a bypass air flow path within the vortex chamber may have on the quality of the sound produced by the vortex chamber. Preferably some of the exhaled air enters the bypass air flow path before the conduit imparts at least a tangential component to air flow entering the vortex chamber.

In a set of embodiments the bypass air flow path is provided at the mouthpiece to immediately divert some of the exhaled air away from the vortex chamber. This is particularly advantageous as it immediately diverts excess exhaled air away from the sound producing part of the device. For example, the device may comprise a separate bypass flow conduit extending from the mouthpiece external to the vortex chamber. Such a bypass flow conduit may guide some of the exhaled air flowing through the mouthpiece directly to a secondary, bypass, outlet.

In a set of embodiments the device may comprise multiple bypass air flow paths. The presence of multiple bypass air flow paths can allow the exhaled air travelling along each bypass air flow path to follow an easier, less restricted path and thus promotes air flow along the bypass air flow paths. This is also particularly advantageous if, for example, a user inadvertently blocks one of the secondary outlets of the bypass air flow paths as air would still be able to pass along another bypass air flow path. In a preferred set of embodiments the device comprises two bypass air flow paths and a separate flow path which supplies the vortex chamber (for example the tangential conduit mentioned above). In various embodiments, these flow paths are provided at the mouthpiece or downstream of the mouthpiece. This ensures that there is always a supply of air to the vortex chamber when the user exhales. The two or more bypass air flow paths are preferably arranged, as described above, such that the exhaled air which leaves the flow dependent sound producing device via the bypass air flow paths produces a secondary sound of substantially constant frequency, such that the secondary sound can be readily distinguished from the primary sound produced by the vortex chamber (which has a frequency dependent on the flow rate).

In a set of embodiments the proportion of exhaled air which bypasses the outlet of the vortex chamber via the bypass air flow path, compared to the volume of exhaled air which enters the flow dependent sound producing device, is dependent on the flow of exhaled air into the device through the mouthpiece. In a preferred set of embodiments the bypass air flow path is selectively opened depending on the volume or flow rate of exhaled air into the device. This may be achieved, for example, by the mouthpiece of bypass flow path comprising an aperture with a restriction element arranged to be moved depending on the volume or flow rate of exhaled air. The restriction element may, for example, be resiliently biased e.g. so that it is only moved when the exhaled air flow applies a minimum counter force. This is advantageous in measuring flow rates of users with both small and large respiratory flow rates. A small flow rate is likely to produce a lower amplitude sound. It is therefore advantageous that when a user with a small flow rate uses the flow dependent sound producing device more air is able to enter the vortex chamber in order to produce a higher amplitude sound. Ensuring that the amplitude of the sound is sufficiently high may be important in ensuring that an electronic device is able to distinguish the sound produced from the flow dependent sound producing device from that of other background noise. In the other extreme, when a user exhales with a strong flow rate it is preferred that a larger portion of that air is able to bypass the vortex chamber, this ensures that the sound produced is not too loud and thus detectable without distortion by the flow dependent sound producing device.

It will be appreciated that the features described above in relation to reducing the amplitude of the produced sound, and producing a secondary sound, may also be provided as features of an apparatus or method according to the first and/or second aspects of the invention defined above. The invention extends to an apparatus for measuring respiratory flow rate comprising: a flow dependent sound producing device as described herein above; an electronic device capable of detecting the sound; a sound analysis module configured to determine a frequency of the sound; and a processor configured to determine the respiratory flow rate from said frequency. Furthermore the invention extends to a method of measuring respiratory flow rate comprising: a user exhaling through a flow dependent sound producing device as described herein above; detecting the sound using an electronic device; and analyzing said sound to determine a frequency of said sound and using said frequency to determine the respiratory flow rate. In embodiments of such an apparatus or method, preferably the electronic device is a mobile device. For example, the electronic device may be selected from a (non-exhaustive) list comprising one or more of: a smartphone, tablet computer, smart watch or other wearable device, personal digital assistant (PDA), or mobile gaming system. In embodiments of such an apparatus or method, the means for reducing the amplitude of the produced sound comprises a bypass air flow path arranged to produce a secondary sound having a substantially constant frequency and the electronic device is arranged to detect the secondary sound as an indicator that a user is exhaling into the flow dependent sound producing device.

There is now described some further features which may apply to embodiments according to any of the aspects of the invention outlined above.

In a set of embodiments the sound producing device comprises a conduit extending between the mouthpiece and the vortex chamber arranged to impart at least a tangential component to air flow entering the vortex chamber. For example the conduit may be tangentially directed. Such a conduit assists in directing the airflow into the vortex chamber so that a strong vortex is produced in the vortex chamber, producing the sound clearly.

In a first set of examples the conduit may be tangentially directed towards the vortex chamber, such that the conduit defines a longitudinal axis extending substantially perpendicular to the axis of the vortex chamber. This means that the net direction of airflow along the conduit is substantially orthogonal to the net direction of airflow through the outlet. Such examples may simplify the design of the device e.g. so that it can be injection molded in a plastics material with minimum complexity and cost involved. In a second set of examples the conduit may be axially directed towards the vortex chamber, such that the conduit defines a longitudinal axis extending substantially parallel to the axis of the vortex chamber. In such examples the conduit may be arranged to impart at least a tangential component to air flow entering the vortex chamber using an airflow guide, for example one or more vanes arranged helically in the conduit to impart a swirling motion to the air flow. This means that the net direction of airflow along the conduit is substantially the same as the net direction of airflow through the outlet. Such examples may involve more complexity in the design and molding of the device, but may be more compact in volume and may assist a user in aiming the outlet towards the mobile electronic device.

The outlet could comprise a simple aperture but in a set of embodiments the outlet pipe comprises an axially extending tube. In a set of embodiments the diameter of the tube is smaller than the diameter of the vortex chamber. An outlet tube with a smaller diameter than that of the vortex chamber may allow a better sound to be produced. In at least some embodiments the axially extending tube is funnel-shaped. This can help to direct air out of the vortex and into the outlet. The funnel-shaped tube may have angled walls or, preferably, curved walls. A curved funnel shape may help to entrain air without creating turbulence.

In a set of embodiments the sound analysis module is incorporated within the electronic device. This is advantageous as it may allow real time feedback to the user on their respiratory flow rate without requiring a network connection. It is not essential however and in another set of embodiments the electronic device is arranged to transmit a sound file to a remote sound analysis module where computation can be performed. This simplifies the processing that is required locally. After the respiratory flow rate is determined, this information can then be transmitted back to the electronic device for display to the user and/or to a third party for evaluation. Preferably the electronic device is a mobile device comprising networking capabilities.

In a set of embodiments the analysis is carried out in real time as opposed to analysing a previously recorded audio clip. This provides the benefit of instantaneously indicating a user's respiratory flow rate, which is beneficial in the instance where a user is blowing into the device thinking they are blowing at full capacity where they are not exhaling at their maximum. An instantaneous read out may indicate that they could perhaps be blowing harder. This therefore saves time recording clips which are not optimum recordings. This method would also allow a sound clip to be recorded once a user has reached a certain flow rate/frequency limit, this therefore means that the sound clip sampled will be concentrated on a period of maximum flow rate rather than the build-up period as the user begins exhaling In a set of embodiments however the sound file may be stored for later download or more detailed analysis, even if a real-time reading has been given. In another set of embodiments the sound file may be discarded after carrying out the analysis, so as to minimize the memory burden on the electronic device. Preferably only the determined respiratory flow rate is stored by the electronic device. Further preferably the electronic device (or its software application) may be arranged to display one or more past measurements of respiratory flow rate in addition to a current measurement of respiratory flow rate. This can assist a user, and others, in recognizing patterns in improvement or deterioration of a user's lung function.

In a set of embodiments the electronic device includes a software application available providing a user interface, processing the sound generated to calculate flow rates and displaying this information to the user, for example displaying respiratory flow rate information to a user. This is advantageous as a user is able to track and monitor their respiratory flow rate over a period of time and if necessary they are able to seek medical assistance based on the analysis.

In a set of embodiments the software application may further act to promote a user's self-awareness and enhance medication compliance. In such embodiments the user interface may be arranged to provide for input by a user of predicted respiratory flow rate. Preferably the user is invited to input a predicted respiratory flow rate before exhaling through the sound producing device to obtain a measurement. The user interface may be further arranged to output a display of both predicted and measured respiratory flow rate. This acts to “gamify” the measurement process so that a user, especially a child, is encouraged to increase their adherence to medication regimes and beat their prediction.

Whether a local software application on the electronic device is used to perform calculations or computation is carried out remotely, there is the possibility of easily and automatically updating the basis used for calculations e.g. as more data is collected and better calibration information is available.

In a set of embodiments the method may further comprise calibrating the sound analysis step or sound analysis module before providing a user with a flow dependent sound producing device. Such calibration may comprise a test process wherein the (typically, substantially linear) relationship between the respiratory flow rate and the determined frequency is verified using a respiratory flow rate meter or spirometer that is different to the flow-dependent sound producing device, i.e. not a vortex whistle. A medical-grade standard peak flow meter may be used for this purpose, for example a Microlife PF100 peak flow meter or a MIR MiniSpir spirometer.

In addition, or alternatively, the sound analysis step or sound analysis module may include input of an identification of the user's flow dependent sound producing device before a measurement is carried out. For example, a software application that is executing the sound analysis process may prompt a user to identify the vortex whistle that is being used, e.g. adult version or child version. Different versions of the flow dependent sound producing device may be optimized for different air flow rates (˜400 lpm for an adult and ˜150 lpm for a child). In particular, an adult version of the flow dependent sound producing device may include the amplitude-reducing means (e.g. bypass air flow path) described above, while a child version of the flow dependent sound producing device may be designed to analyze sounds having a naturally lower amplitude. In such a child version the amplitude-reducing means (e.g. bypass air flow path) may be absence or blocked from operating.

In a set of embodiments the mouthpiece is oval in shape which makes it more comfortable to use. Alternatively it could be circular or another suitable shape. In a set of embodiments the mouthpiece comprises a lipped section, this allows the user to position their mouth over the mouthpiece and achieve a good seal. This is advantageous as it ensures that all of the air being exhaled by the user is transferred into the sound producing device thus allowing more accurate results. The mouthpiece could comprise a flexible flange portion and/or teeth grips (e.g. in the manner of the mouthpiece on a snorkel) to allow a better seal between the user and the device, again for more accurate measurement of flow rate.

The flow dependent sound producing device could be attached to the mobile device e.g. through the use of a sleeve or clip. This might be beneficial in optimizing transfer of sound to the microphone of the electronic device and ensuring consistency in the measurements because the device is always the same distance from the microphone. In another set of embodiments however the flow dependent sound producing device is separate from the mobile device.

In a set of embodiments the mouthpiece is removable for cleaning or to allow interchangeability if the device is to be used by several people. In a further set of embodiments the flow dependent sound producing device is modular and can be constructed from a number of components. In a preferred set of embodiments the flow dependent sound producing device is produced from four components: a mouthpiece, a top half, a bottom half, and an outlet portion. Such a modular construction would allow a user to completely disassemble the flow dependent sound producing device allowing them to thoroughly clean the device. A modular construction may also make manufacture of the flow dependent sound producing device easier.

In a preferred set of embodiments a cap is provided to cover the mouthpiece. This is advantageous as it shields the mouthpiece thus protecting it from external contaminants Subsequently this would allow the user to store the fluid transfer device on their person without being concerned that it would become contaminated.

In a set of embodiments the flow dependent sound producing device is handheld. In a set of embodiments the flow dependent sound producing device is made from a plastics material, for example polyurethane or PVC. This ensures that it is lightweight and allows it to be held in a user's mouth without need for external support.

In a set of embodiments a removable sleeve is provided which surrounds the flow dependent sound producing device. In a preferred set of embodiments, one or more complementary features are provided on the flow dependent sound producing device and incorporated into the removable sleeve. This helps to hold the removable sleeve in position on the flow dependent sound producing device. In one set of embodiments, such a removable sleeve may be made of a flexible (e.g. elastomeric) material and slid over the device. In another set of embodiments, such a removable sleeve may be made of a rigid (e.g. thermoplastic or thermoset) material and clipped onto the device.

In various embodiments the removable sleeve has decorative features that would make it aesthetically pleasing to the user. This may for example include decorations to make it appear like a fish. The use of a removable sleeve is beneficial as it provides an aesthetically pleasing finish to the flow dependent sound device. This may be particularly relevant when it comes to encouraging younger children to use the device. In various embodiments the removable sleeve is made from a soft thermoplastic material, such as silicone, or a hard thermoplastic material. The thermoplastic material may be chosen so as to provide the user with a better grip of the device and/or to provide a protective function.

The mobile electronic device is preferably a handheld device such a smartphone, tablet computer, smart watch or other wearable device, personal digital assistant (PDA), or mobile gaming system—this makes taking recordings easier as the device can be held close to the sound producing device which is in a user's mouth.

The frequency of the detected signal may be determined e.g. by taking the Fast Fourier Transform (FFT) of the signal. Where the detected sound contains a number of frequencies, the frequency used to determine the flow rate could be established in a number of ways including but not limited to: the maximum, median or mean frequency, frequency with highest power or root mean square power, center of the 3 dB band etc.

Any reference herein to measuring or determining respiratory flow rate may be taken to include measuring or determining peak expiratory flow rate (PEFR or PEF) or “peak flow”.

In various embodiments the methods and apparatus described herein may further comprise using the determined frequency of the (primary) sound produced by the vortex chamber to determine one or more further parameters relating to the respiratory flow rate. Such parameters may include one or more of: Forced Vital Capacity (FVC); Forced Expiratory Volume in 1 Second (FEV1); and the ratio FEV1/FVC. When a user exhales through the flow dependent sound producing device, the exhaled air flow rate (L/min) is determined from the frequency of the (primary) sound produced and the way that the respiratory flow rate changes with time can be used to determine these parameters. For example, FVC is measured as the total volume of exhaled air. FEV1 is measured as the volume of air exhaled during the first second of exhalation. FEV1 is a frequently used index for assessing airway obstruction, bronchoconstriction or bronchodilatation e.g. due to asthma or other obstructive lung disorders.

### BRIEF DESCRIPTION OF THE DRAWINGS

Certain embodiments of the invention will now be described, by way of example only, with reference to the accompanying drawings in which:

FIG. 1 shows a view of a flow dependent sound producing device in accordance with an embodiment of the invention;

FIG. 2A shows a side view of the flow dependent sound producing device of FIG. 1;

FIG. 2B shows a section of the device along line B . . . B

FIG. 3 shows schematically the sound producing device being used with a mobile device capable of recording the sound;

FIG. 4 shows a graph showing the relationship between flow rate and frequency;

FIG. 5 is a flow diagram of the process involved in producing and analyzing the sound;

FIG. 6 shows schematically an alternative embodiment of the flow dependent sound producing device;

FIG. 7 shows a view of a flow dependent sound producing device i.e. a vortex whistle in accordance with another embodiment of the invention;

FIGS. 8A-8D show the various components of the vortex whistle shown in FIG. 7;

FIG. 9 shows an internal view of the vortex whistle along the line A-A in FIG. 7;

FIG. 10 shows the vortex whistle of FIG. 7 provided with a ‘plain’ external sleeve;

FIG. 11 shows the vortex whistle of FIG. 8 provided with a ‘fish’ shaped external sleeve;

FIG. 12 is another flow diagram of the process involved in analyzing the sound;

FIGS. 13A-13D are examples of a user interface displayed by an app before a sound analysis trial; and

FIGS. 14A-14C are examples of a user interface displayed by an app during and after a sound analysis trial.

### DETAILED DESCRIPTION OF THE INVENTION

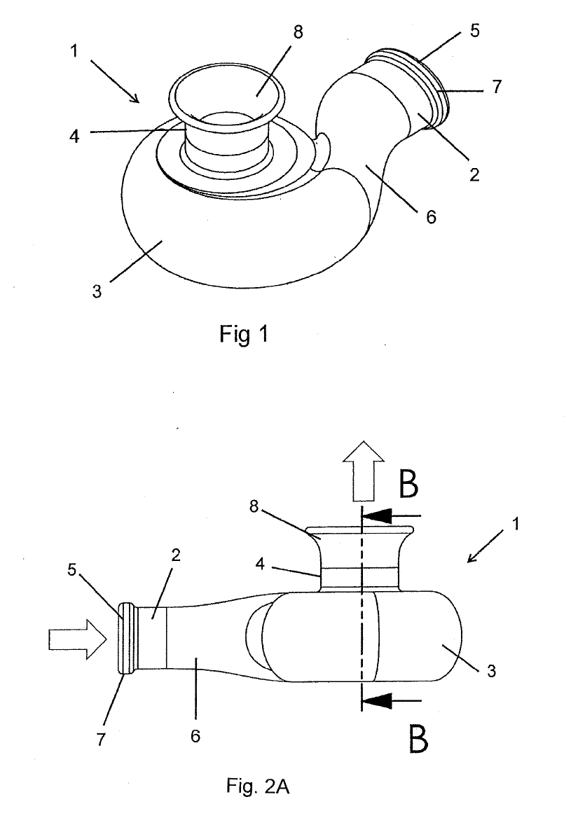

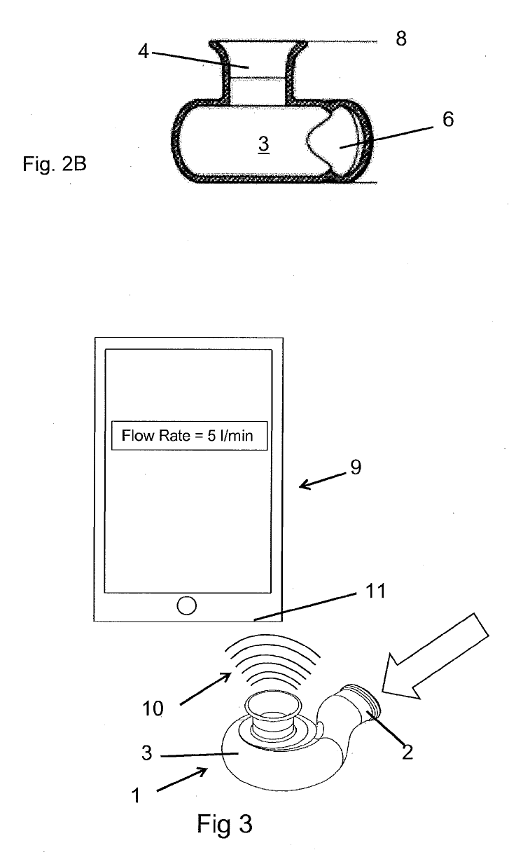

FIGS. 1, 2A and 2B show a flow dependent sound producing device 1 in accordance with an embodiment of the invention. The device has a mouthpiece 2, a vortex chamber 3 and an outlet tube 4. The mouthpiece 2 has an oval shaped opening 5 which tapers into a circular-section spiral tube 6 leading into the vortex chamber 3. The mouthpiece 2 also comprises a ridged section 7, which allows a user to position his/her mouth over the pipe, creating a strong seal. The oval-shaped mouthpiece 5 may have a minor axis in the range of 8-20 mm, e.g. 10-15 mm.

The outlet tube 4 has a circular cross section and extends in a direction away from the vortex chamber 3 along its axis (shown by the dashed line B . . . B in FIG. 2A). The outlet tube 4 flares into a horn shaped mouth 8, starting with a smaller diameter close to the vortex chamber 3 and extending to a larger diameter further from the chamber 3. The outlet tube 4 may have a minimum diameter in the range 5-30 mm, e.g. 10-20 mm.

The vortex chamber 3 is a flat cylindrical shape with a curved edge. The base of the chamber 3 to the top of the outlet pipe 4 is preferably in the range 20-80 mm, e.g. 30-60 mm. The opening of the mouthpiece 2 to the far edge of the vortex chamber 3 is preferably in the range 50-100 mm.

As seen in FIGS. 2A and 2B, the diameter of the vortex of air changes abruptly from the larger diameter in the vortex chamber 3 to the smaller diameter of the outlet pipe 4. This is not necessarily required and this change can be performed gradually by a tapered entrance into the outlet tube 4.

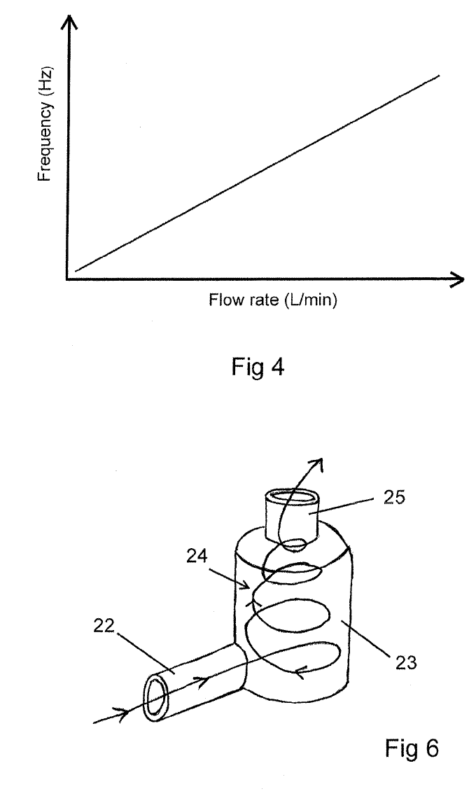

FIG. 3 illustrates the use of the flow dependent sound producing device 1 with a smartphone device 9. The sound 10 produced by the sound producing device 1 is detected using a microphone 11 forming part of the smartphone 9. Of course, the smartphone 9 could be replaced by any other mobile digital device having a built-in microphone, such as a smart watch or other wearable device, tablet, personal digital assistant (PDA), mobile gaming system, etc. Upon detecting the sound 10, the smartphone 9 converts it into an electronic signal. This signal is analyzed using the on-board processor and may also be recorded as an audio clip. This audio clip can subsequently be interpreted by the processor of the smartphone 9 at a later time or downloaded for analysis elsewhere. The signal is analyzed to determine a characteristic frequency (as will be explained in more detail below) which can then be converted into a flow rate using a relationship such as the graph of FIG. 4.

FIG. 4 shows the characteristic relationship between the flow rate of air into the device 1 and the measured frequency of the sound 10 produced as a result of the exhaled air passing through the vortex chamber 3. This graph demonstrates the linear relationship which is converted into a lookup table stored in the memory of the smartphone 9. This allows the flow rate to be calculated from the measured frequency. It will be appreciated that, as a user exhales through the mouthpiece 2, the flow rate generally falls from a peak flow rate towards a zero value as the lungs are emptied of breath. This means that the sound 10 produced by the vortex chamber 3 starts at a high frequency and gradually changes to a lower frequency as the flow rate is reduced. This change in frequency can be measured by the processor of the smartphone 9 and used to determine how the flow rate varies during an exhalation cycle. The flow rate, and in particular the way that the flow rate changes in time, may be used to determine one or more further parameters such as FEV1, FVC or FEV1/FVC.

In use, a user places his/her mouth around the mouthpiece 2 and exhales. Air flows through the mouthpiece 2 and tangentially into the vortex chamber 3 establishing a circulating vortex in the chamber 3. The air rotates around the axis of the chamber 3 in a spiral towards and out through the outlet tube 4. As the air passes into the smaller diameter outlet tube 4, the speed of rotation of the air increases according to the law of conservation of angular momentum.

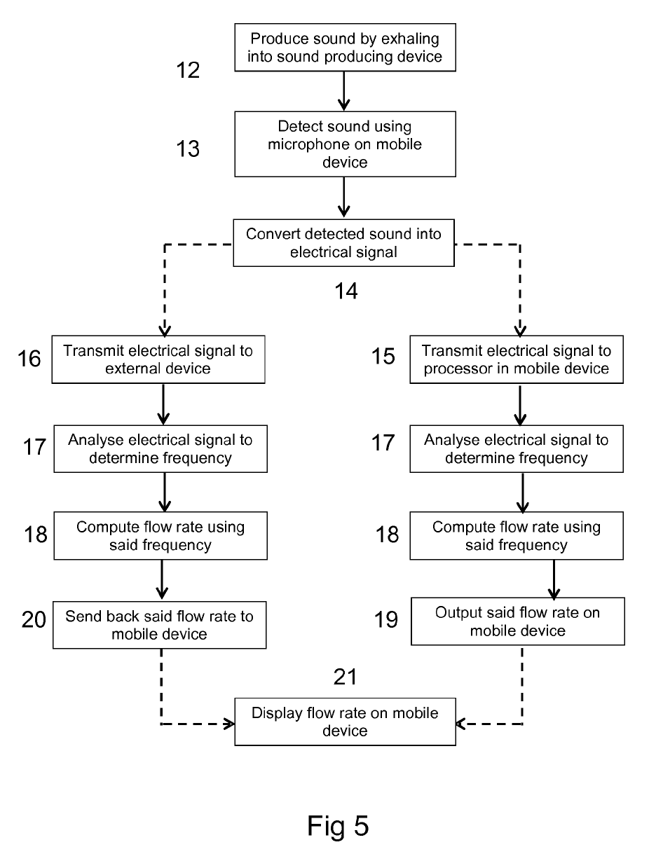

As the air passes out of the outlet tube 4 it produces an audible sound 10 which travels towards the microphone 11 on the mobile e.g. smartphone device 9 (steps 12 and 13, FIG. 5). The microphone 11 converts this sound into an electrical signal (step 14, FIG. 5) which can then be transmitted to the processor on the mobile device (step 15, FIG. 5). This is then analyzed by the processor in the mobile device 9 to calculate the frequency (step 17, FIG. 5). This can be achieved by a number of methods including but not limited to the Fast Fourier Transform (FFT) of the detected spectrum. The sound detection process may use frequency-domain analyses, some time-domain analyses, and high- and low-pass frequency filters in order to clean the signal. The flow rate is calculated using calibration data of frequency versus flow rate as seen in FIG. 4 (step 18, FIG. 5). It is possible to determine this using a mathematical function or a look-up table. The flow rate is then displayed on the mobile device (steps 19 and 21, FIG. 5). The flow rate may be recorded and/or compared to previous flow rates for the user or typical flow rates for a user of set parameters such as age, height and weight. One or more further parameters, such as such as FEV1, FVC or FEV1/FVC, may also be displayed.

As discussed previously the computation of the frequency from the sound clip does not necessarily have to be carried out on the mobile device itself. This can be performed on an external device following the steps in the left hand branch of FIG. 5. This includes transmitting the recorded sound to the external device (step 16) analyzing the sound to compute the frequency (step 17), computing the flow rate from said frequency (step 18), and then returning this flow rate back to the mobile device (step 20). This flow rate can then be displayed on the mobile device (step 21).

For optimum readings the microphone 11 on the mobile device 9 should be positioned in close proximity to the outlet mouth 8, as this increases the signal to noise ratio of the detected sound 10. As will be explained further below, the device may include means for reducing the amplitude of the sound produced e.g. in the event of the sound being so loud as to cause distortion in it detection or disturbance to a user.

In the embodiment discussed so far the sound producing device 1 has a curved inlet pipe 6 leading into the vortex chamber 3 which continues this curved path. In another embodiment, shown in FIG. 6, the inlet pipe 22 leads directly into the vortex chamber 23 where the vortex chamber 23 which in this embodiment has a taller, cylindrical shape. The vortex 24 is established and accelerates through the outlet 25 as before.

In a further alternative embodiment a flatter cylindrical chamber could be provided, which may make it easier to carry for instance in a user's pocket. The vortex chamber and inlet pipe could be of any shape so long as when the air passes into the device it forms a vortex and is able to escape and produce a sound.

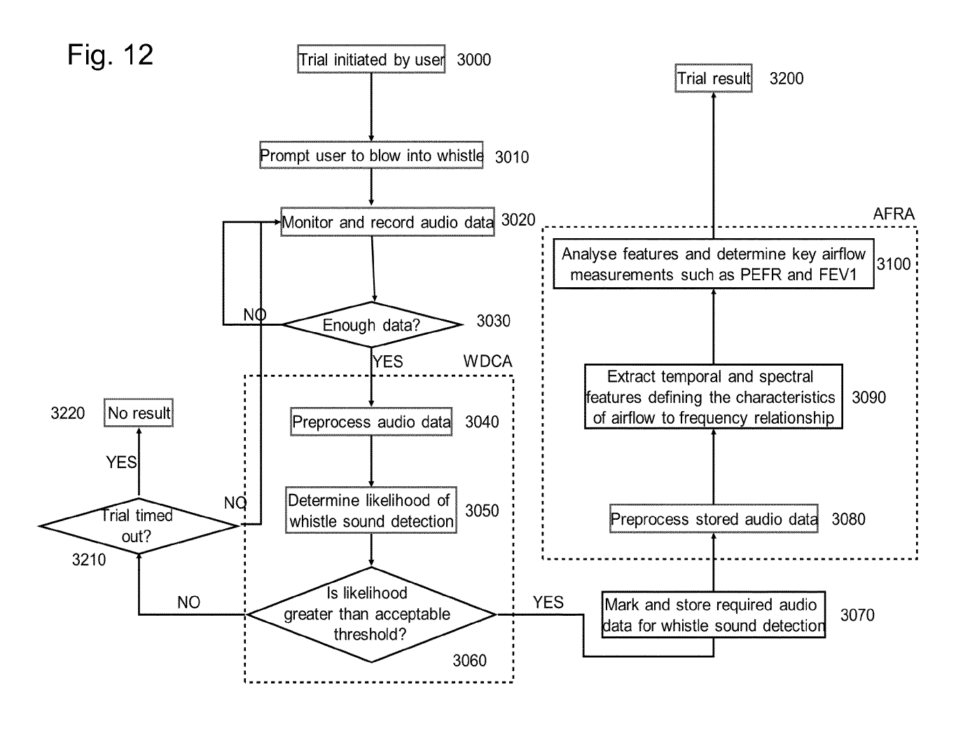

Each sound producing device can be calibrated during production and its own individual look-up table or mathematical function generated to ensure its accuracy. FIG. 12 provides a more detailed flow diagram for a typical software process executed by the processor of a mobile digital device with audio input and networking capabilities (such as a mobile phone, wearable device e.g. smart watch, personal digital assistant, mobile gaming system, or tablet). Steps 3080-3100 relate to the airflow rate and frequency relationship algorithm (AFRA). Before a particular sound producing device 1 is released, the AFRA is trained on an audio data set including real whistle sounds with known peak flow values. Typically a user will inform the software process about which device is being used, e.g. the associated app will enable a user to select their device type from a list. The training process will go through the same steps as 3080-3100 and will be conducted prior to releasing the app. The airflow rate and frequency relationship may be calibrated during the training process using one or more different standard spirometers, for example a Microlife PF100 peak flow and FEV1 meter or a MIR MiniSpir spirometer. Any well-made medical grade spirometer may be used as a calibration standard for the sound producing device and related app.

The stages of the software process seen in FIG. 12 are as follows. In stage 3000 the software process is initiated by manual user input (such as pressing a button, or a virtual touch-screen button, on the mobile device). In stage 3010, the software process communicates to the user that it is ready to receive audio input (in other words, that it is ready for the user to blow into the whistle 1). In stage 3020, the process monitors audio input, and records audio data in preparation for detection of valid whistle sounds. In stage 3030, the software process determines if enough audio data is recorded for use in the whistle detection classification system (see stage 1000). Stage 3040-3060 defines the whistle detection classification algorithm (WDCA). The WDCA is trained to recognize whistle sounds on an audio data set including real whistle sounds and to reject non-valid sounds like speech and music, etc. The WDCA will calculate similarity metrics to determine the likelihood of a valid whistle sound which can be used to better assist the user in producing a valid whistle sound. The training process will go through the same steps as stage 3040-3060 and will be conducted prior to releasing the app.

In stage 3040, the software process pre-processes the audio data using time- and frequency-based filters to remove unnecessary elements in the audio data and extract time- and frequency-based features for use by the whistle detection classification algorithm (WDCA). In stage 3050, the software process calculates the likelihood of a whistle sound based on the trained pre-processed audio data and feature extraction from stage 3040. In stage 3060, the software process determines whether the likelihood from stage 3050 is above a certain threshold for accepting the whistle sound as valid. In stage 3070, the software process marks and stores the audio data used to determine a valid whistle sound.

Stage 3080-3100 defines the airflow rate and frequency relationship algorithm (AFRA). As described above, the AFRA is trained on an audio data set including real whistle sounds with known peak flow values. The training process will go through the same steps as stage 3080-3100 and will be conducted prior to releasing the app. In stage 3080, the software process pre-processes the audio data marked and stored in stage 3070 using time- and frequency-based filters to filter out unnecessary elements in the audio data. In stage 3090, the software process extracts temporal and spectral features defining the characteristics of the airflow rate and frequency relationship. In stage 3100, the software process analyses the features extracted in stage 3090 and determines key airflow measurements such as PEFR and FEV1 based on the trained airflow rate and frequency relationship algorithm (AFRA).

In stage 3200, the software process arrives at results for the trial (including the key respiratory metrics determined in stage 3100) and makes these results accessible to entities outside of the software process, such as the user and other software processes.

In stage 3210, the software process determines whether a trial has continued for longer than a certain maximum allowed duration. In stage 3220, the process communicates to the user that the trial has timed out and provides the user with relevant feedback of a corrective, instructional, and/or motivational nature based on the likelihood determined.

The trial result produced at stage 3200 may be displayed on the mobile device and stored locally and/or remotely e.g. in the cloud. After each trial, the audio data that was stored for use in the AFRA stage may be discarded so as to minimize the memory requirements of the app. A mobile device that executes such a software process is advantageously able to carry out a real-time analysis and provide a user with a rapid result that includes meaningful information such as a user's PEFR,

In the embodiment described above the sound producing device 1 is formed from two injection molded plastic components (e.g. in a clamshell design). Equally however it could be manufactured as separable components: e.g. the mouthpiece 2 could come in different shapes and sizes dependent on its specific application for instance for children or adults. These components could be held in place by screw means or snap fittings with rubber seals.

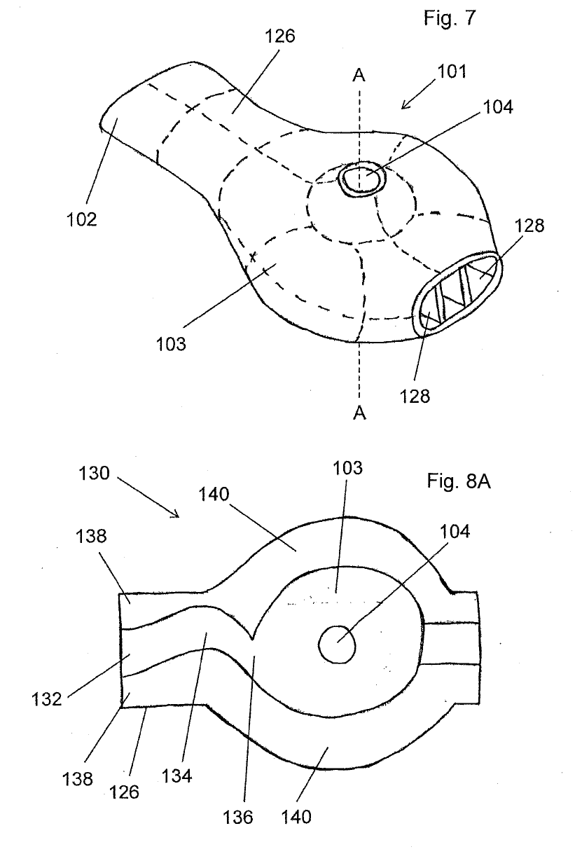

FIG. 7 shows an alternative embodiment of the flow dependent sound producing device i.e. vortex whistle 101. The vortex whistle 101 comprises a mouthpiece 102 and an outlet tube 104 similar to the vortex whistle of FIG. 1. The mouthpiece 102 is connected to an elongate portion 126 which is connected to the vortex chamber 103. The notable difference with the vortex whistle 101, compared to that of FIG. 1, is that within the main body there is incorporated two bypass air flow paths which have corresponding bypass outlets 128. Such bypass air flow through the vortex whistle 101 will be explained with reference to the later Figures. Embodiments of the flow dependent sound producing device which comprise bypass air flow paths are particularly well-suited for adult users who produce higher air flow rates. The same devices may be provided to child users with the bypass air flow paths absent or blocked.

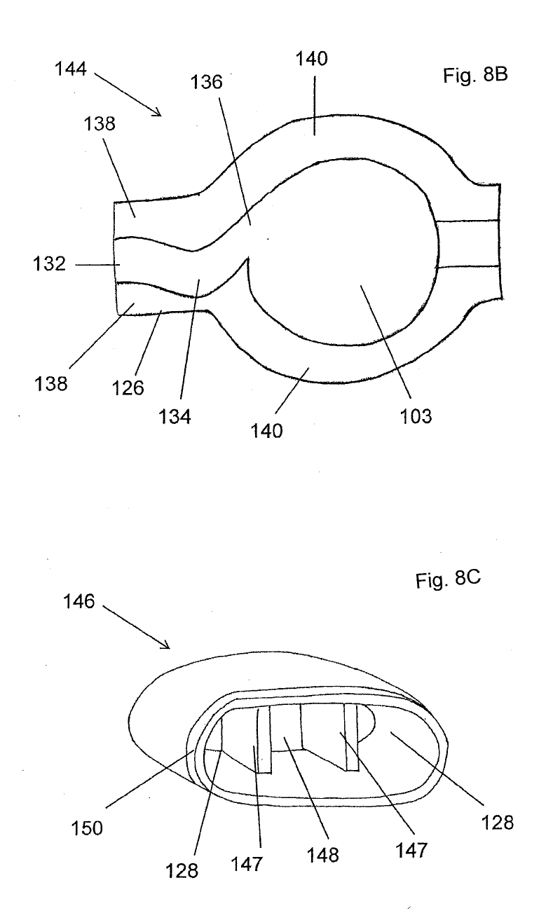

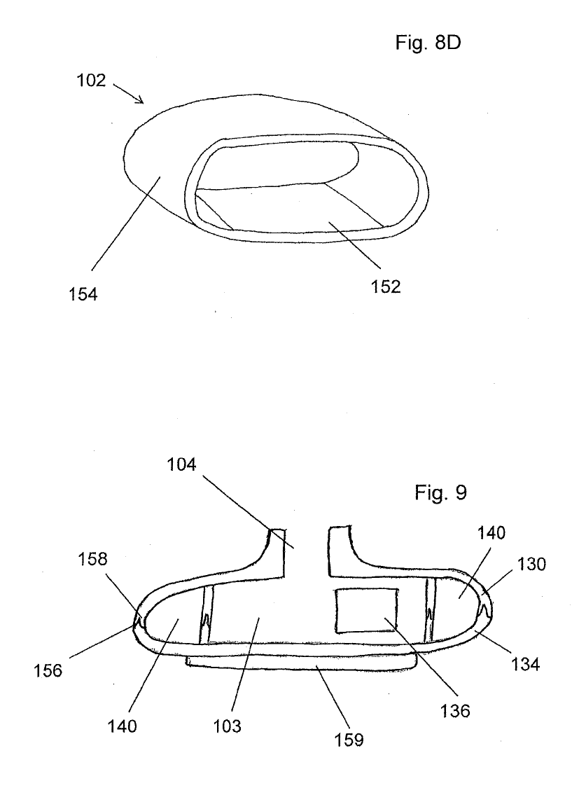

The vortex whistle 101 is comprised of four components: the main body is split into two halves 130, 144 and then there is a mouthpiece 102 at one end and a bypass outlet piece 146 at the other end. The four components are all secured together to form the vortex whistle 101. The four components can be seen in FIGS. 8A-D. The main body of the vortex whistle 101 is formed from two halves 130, 144 extending in a plane perpendicular to the axis of the vortex chamber 103. FIG. 8A shows an internal view of the top half 130 of the vortex whistle 101. The top half 130 has internal walls which form vortex the chamber 103 and form the outlet tube 104. In the elongate portion 126 of the top half 130 it can be seen that there are three internal pathways. One pathway 132 acts as an inlet for the vortex chamber 103. This pathway 132 has an elongate portion 134 which curves within the top half 130 such that it enters the vortex chamber at point 136 in a direction which is tangential to the flow around the axis of the vortex chamber 103. The introduction of tangential air flow helps in establishing a vortex.

Also shown in FIG. 8A are two bypass air flow path inlets 138 in the elongate portion 126. These direct air into bypass air flow channels 140 separated from the vortex chamber 103 by internal walls. The bypass air flow channels 140 pass around the outer circumference of the vortex chamber 103 and allow exhaled air to pass out of the corresponding bypass outlets 128 in the bypass outlet piece 146 (seen in FIG. 8C). As previously discussed, the bypass air flow channels 140 allow some of the air exhaled by the user to bypass the vortex chamber 103. This is advantageous as it reduces the amplitude of the sound produced from the vortex whistle 101, which otherwise may cause the sound produced to be too loud for accurate analysis and/or user comfort. Furthermore, the bypass air flow channels 140 may produce a secondary sound, in particular a constant frequency sound, that can be detected by the microphone 11 of a smartphone 9 (see FIG. 3) and used to identify when the vortex whistle 101 is being used. This can assist the processor of the smartphone 9 in recognizing the primary sound 10 produced by the vortex chamber 103 and distinguishing this sound 10 from background noise.

FIG. 8B shows the lower half 144 of the vortex whistle 101. It can be seen that the lower half 144 comprises complementary internal walls to that of the top half 130 such that when the lower half 144 it is placed against the top half 130 the vortex chamber 103, inlets 132, 138 and bypass air channels 140 are sealed inside the main body. As discussed with reference to the other vortex whistle of FIG. 1, the closed base directs air which enters the vortex chamber 103 and forms a vortex to travel upwards and pass out the outlet 104 and thus produce a sound.

FIG. 8C shows the bypass outlet piece 146. The bypass outlet piece 146 is formed from a single piece of plastic. It comprises three separate distinct portions Ruined by internal vertical walls 147. The vertical walls 147 form the two bypass outlets 128 which allow air that has flowed through the bypass air channels 140 to leave the vortex whistle 101. Also shown is a central portion 148. The purpose of the outlets 128 is to direct the flow from the bypass air channels 140 away from the vortex whistle 101. The walls 147 are provided so as to divert the escaping air such that each bypass air flow path does not come into direct contact with the other. This can help each bypass air channel to produce a secondary sound of fixed frequency. Similar to a standard whistle, either or both of the bypass air channels 140 may include a hole (not shown) in an outer wall of the whistle 101 that is shaped/angled to set the frequency of such a secondary sound. In the embodiment shown the central portion 148 does not function to allow air to escape. It is however appreciated that in certain situations this separation via the walls 147 may not be necessary, however the presence of such walls 147 does add rigidity to the bypass outlet piece 146. The outlet piece 146 further comprises a ridged portion 150 extending around its periphery, the relevance of this will be explained in later Figures.

FIG. 8D shows the inlet mouthpiece 102. The inlet mouthpiece 102 comprises a hollow section 152 contained by an outer shell 154. The inlet mouthpiece 102 is the component which a user exhales into when using the vortex whistle 101. The hollow section 152 directs the exhaled air towards the vortex inlet 132 and bypass inlets 138 in the elongate portion 126 of the main body. Although not shown, there may be provided a closed cap which can be placed over this inlet mouthpiece 102. This would act to prevent contact of the inlet mouthpiece 102 with any unclean surfaces or contaminants It is also envisaged that the inlet mouthpiece 102 could also comprise internal walls similar to the bypass outlet piece 146 to immediately establish the three separate air flow paths.

FIG. 9 shows a cross-section of the vortex whistle 101 through the line A-A shown in FIG. 7. This cross section shows how the bypass air channels 140 pass around the circumference of the vortex chamber 103. Additionally the cross section shows how the top half 130 and lower half 144 are connected together. The internal walls on the top half 130 are provided with a groove 158 that is designed to receive a protrusion 156 provided on internal walls of the lower half 144. The groove 158 and protrusion 156 extend around the entirety of the walls provided on the top half 130 and lower half 144 both internally and externally. The protrusion 156 and groove 158 may be designed such that a friction fit may be sufficient to hold them together. This may be achieved by making the groove 158 slightly smaller than the protrusion 156 such that when the two halves 130, 144 are pressed together the protrusion 156 causes the groove 158 to expand around the protrusion 156 and grip onto it. Such embodiments may be advantageous as they may allow the user to dismantle the whistle enabling them to clean or change components of the whistle. In such embodiments the two halves 130, 144 may be made from plastic to allow the grooves to flex sufficiently to allow the friction fit to function.

In alternative embodiments it may be possible to provide glue between the protrusion 156 and slot 158 in order to permanently fix the two halves 130, 144 together. It is also appreciated that other means may be provided for connecting the two halves 130, 144 together. Once the two halves 130, 144 are connected together to form a main body of the vortex whistle 101, the mouthpiece 102 and bypass outlet piece 146 can be attached at either end. The mouthpiece 102 and bypass outlet piece 146 may be made of the same or different plastic material to the main body. Of course, in other embodiments the mouthpiece 102 and bypass outlet piece 146 may be integrated with the two halves 130, 144 rather than attached as separate pieces.

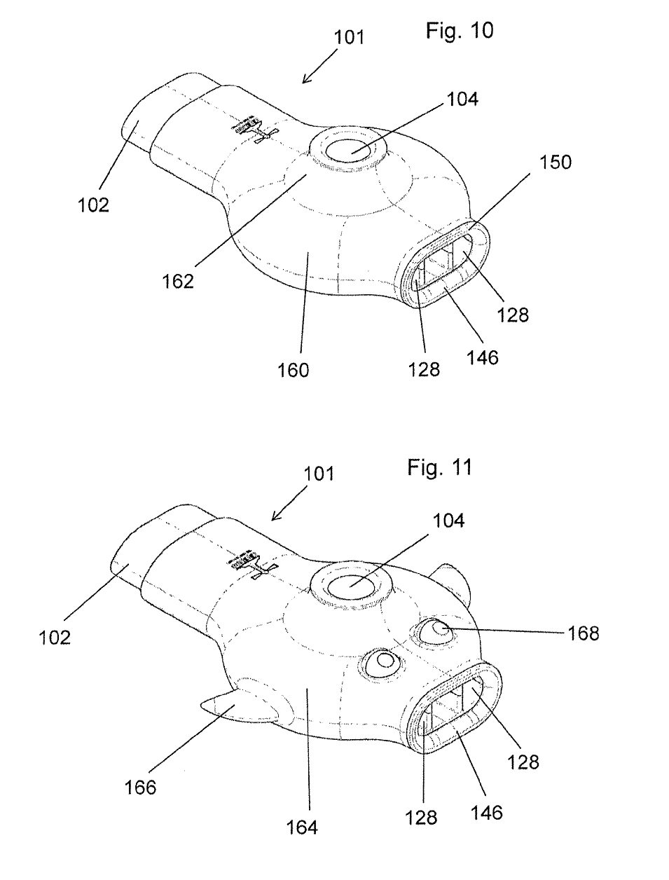

Also visible in FIG. 9 is a lower ridged portion 159 which is provided on the base of the lower half 144 of the vortex whistle 101. The purpose of this ridged portion 159 is to assist in holding an outer sleeve around the vortex whistle 101. This will be explained in more detail with reference to FIGS. 10 and 11.

FIG. 10 shows an embodiment of the ‘plain’ vortex whistle 101. In this embodiment the vortex whistle 101 is covered by an outer sleeve 160. The outer sleeve 160 forms a shell which has a similar shape to the underlying vortex whistle 101, this allows the outer sleeve 160 to be pushed onto the whistle over the mouthpiece 102. The outer sleeve 160 fauns around the bypass outlet piece 146 and the funnel shaped portion 162 which forms the outlet tube 104. The sleeve 160 abuts against the rim 150 on the bypass outflow piece 146 which prevents the sleeve 160 from sliding off the vortex whistle 101. The sleeve 160 contains a cut out portion which slots over and around the ridged portion 159 provided on the base of the vortex whistle 101 (as seen in FIG. 9). This acts to hold the sleeve 160 in place and requires the user to physically move the outer sleeve 160 over the ridged portion 159. The sleeve 160 may be made from a range of thermoplastic or elastomeric materials, however one suitable material is silicone, which allows the sleeve 160 to be easily slid onto the vortex whistle 101. A silicone sleeve would also provide added grip for the user. In other embodiments the sleeve 160 may be made from a harder thermoplastic material and clipped onto the underlying whistle 101, e.g. in two or more parts.

FIG. 11 shows an alternative embodiment called the ‘fish’ vortex whistle. In this embodiment a sleeve 164 similar to that in FIG. 10 is provided on the vortex whistle 101. The sleeve 164 has incorporated fins 166 and eyes 168 so that the vortex whistle 101 has an appearance similar to that of a fish. The bypass outlets 128 look like a fish mouth. The appearance of the ‘fish’ vortex whistle 101 is advantageous as it may encourage children to use the vortex whistle 101. Although in the embodiment shown the sleeve 164 is shaped to look like a fish it is appreciated that there may be various other designs which could be produced. A significant advantage of the sleeves 160, 164 is that they can be changed for alternative sleeves. For example, as a child grows older and no longer desires the ‘fish’ vortex whistle they are able to change it for an alternative design, for example the ‘plain’ sleeve 160. This means that the user does not need to replace the entire device and can simply replace the outer sleeve 160, 164 which is a more cost effective solution.

It can be seen from FIGS. 10 and 11 that a removable cover (not shown) may fit over the mouthpiece 102 to prevent dirt from entering the whistle 101 when not in use.

In the embodiments shown in FIGS. 7-11 there are two bypass air paths provided, one either side of the vortex chamber 103. It is appreciated that in alternative embodiments there may be provided alternative arrangements of bypass air paths for example a single bypass air path may be provided. It is also appreciated that there may be more than two bypass air paths. Furthermore, in the embodiments shown the inlets 138 for the bypass air path are positioned next to the inlet 132 for the vortex chamber, however it is envisaged that alternative arrangements may also achieve the same effect. For example the vortex chamber inlet 132 may contain an aperture part way along its length which allows some air to escape after entering the vortex chamber inlet 132 but before entering the vortex chamber 103. Alternatively (or in addition) an additional outlet may be provided in the vortex chamber 103 to permit the outflow of some air through a bypass outlet rather than the axial outlet 104, as long as the main vortex is not unduly disturbed.

Furthermore, although in the embodiments shown the bypass outlet piece 146 is comprised of two vertical walls 147 which divide the bypass outlets 128 it is appreciated that this may not be necessary, and the two bypass air paths 140 may combine at this point so that air exits through a single bypass outlet. Additionally, in the embodiments shown the vortex whistle 101 is comprised of four separate e.g. plastic components however this is not necessary and for example it may be possible to produce the whistle as a single component, for example from a single piece of injection molded plastic. It is appreciated that the vortex whistle 101 could be formed of various materials, for example one or more of: metal, plastic, wood or a composite material.

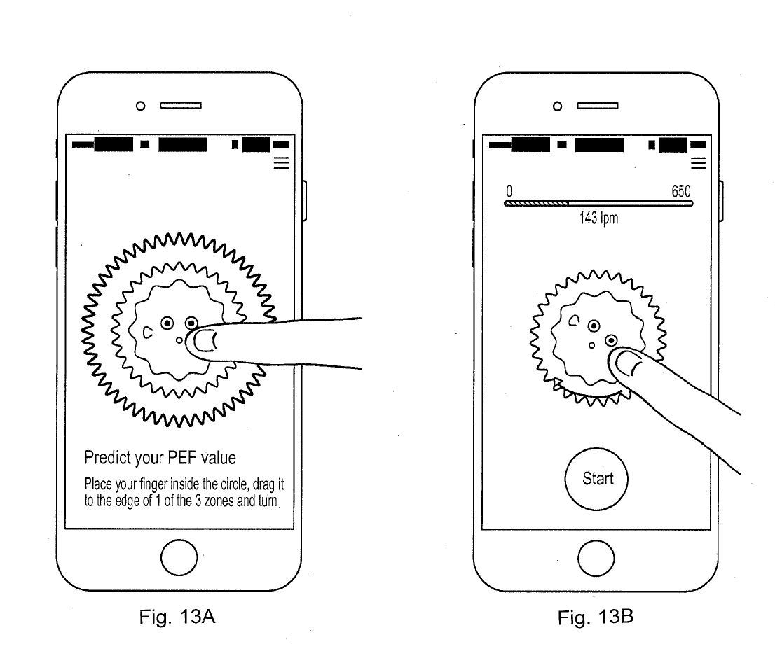

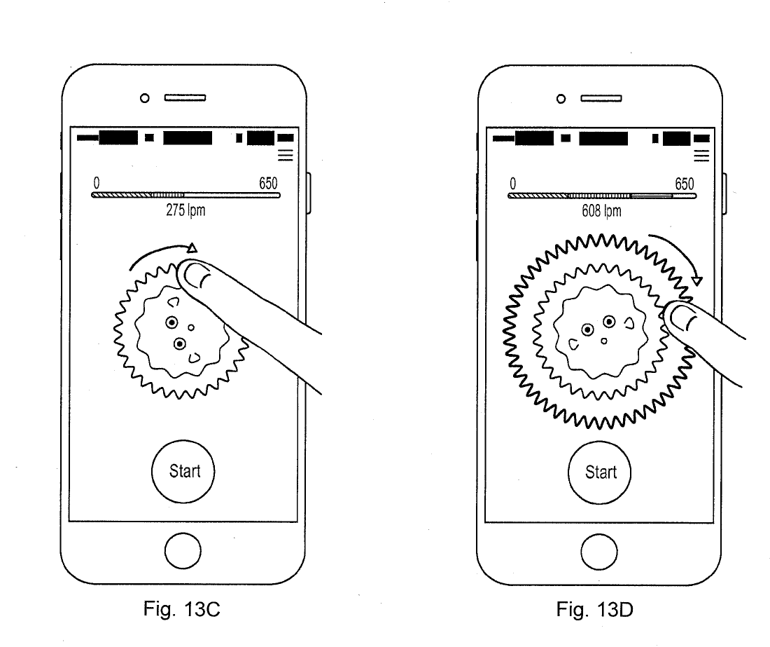

FIGS. 13A-13D provide examples of a user interface displayed by an app that is used in conjunction with any of the sound producing devices i.e. vortex whistles described above. When a trial is initiated by a user, the app first of all prompts the user to predict his or her PEFR value. Research shows that children who guess their PEFR before seeing their actual PEFR have an improved ability to perceive asthma symptoms and this increases their adherence to controller medications for asthma. The app displays an icon which resembles a blowfish inflating. Just as a blowfish inflates itself for protection, so must the user blow into the whistle in order to expand the blowfish icon. The prediction stage appeals to the competitive side of users, as you need to improve your latest “score” and beat your prediction. The blowfish icon can simply be adjusted like a dial so that it is fast and easy to use. Asking users to register their subjectively assessed lung function (PEF) before each measurement facilitates better self-awareness of the symptoms in addition to “gamifying” the process, which will in turn increase user adherence.

As seen in FIG. 13A, the user interface first invites a user to predict today's PEF (peak expiratory flow rate) value. As seen in FIGS. 13B, 13C and 13D, the user then slides his/her finger around the dial shape of the blowfish icon to predict how high the PEF value will be. The predicted PEF is displayed in liters per minute (lpm) as a value on a colored scale across the top of the user interface. Once the user has finished the prediction stage, he/she presses the Start button to initiate the trial itself and then blows into the whistle.

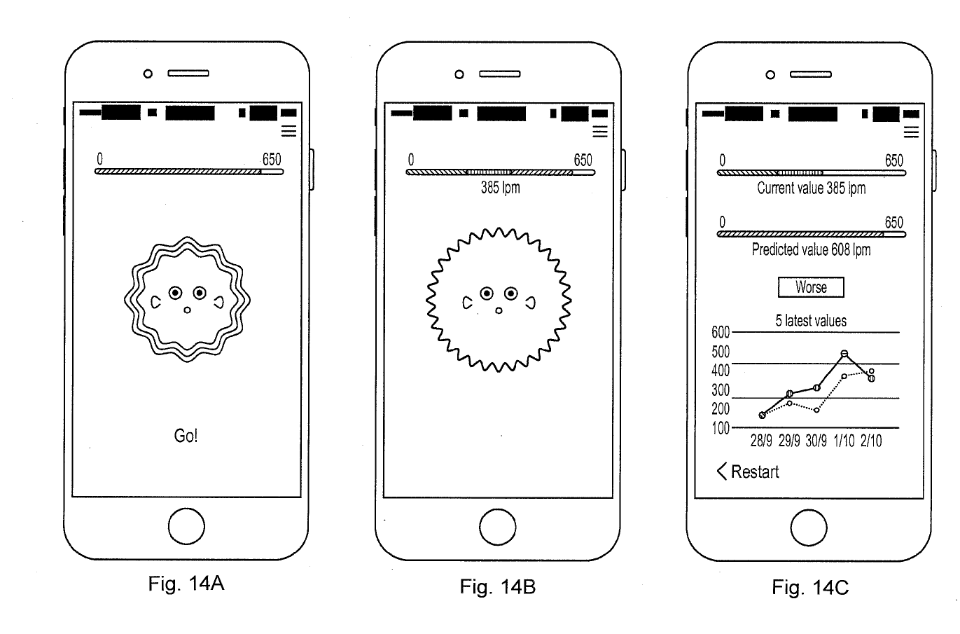

FIGS. 14A-14C provide examples of the user interface displayed by the app during a trial. The initial screen shown in FIG. 14A displays the user's predicted PEF value on the bar at the top and the blowfish icon, which starts small in size, bounces as an encouragement for the user to start the trial. As the user exhales into the whistle, the screen seen in FIG. 14B is displayed, with the blowfish icon expanding in size and the calculated PEF value is compared to the predicted value on the top bar. After each trial, a results detail screen as seen in FIG. 14C may be displayed, which includes a graph comparing the five latest measured and predicted PEF values. This information may be transmitted to an external server and accessible in the cloud by parents and/or healthcare professionals. A further screen may display a user's history based on the most recent measurements, or measurements from the last week or two, or more. This may include both measured values and predicted values.

What is claimed is:

1. A method of measuring respiratory flow rate comprising:
a user exhaling through a flow dependent sound producing device which comprises a mouthpiece and a vortex chamber having an axis and an outlet such that exhaled air flows through the mouthpiece into the vortex chamber causing the exhaled air to form a vortex around the axis and then pass out of the chamber via the outlet in an axial direction, thereby producing a sound;
detecting the sound using a mobile electronic device; and
analyzing said sound to determine a frequency of said sound and using said frequency to determine the respiratory flow rate.

2. An apparatus for measuring respiratory flow rate comprising:
a flow dependent sound producing device comprising a mouthpiece and a vortex chamber having an axis and an outlet such that in use exhaled air flowing through the mouthpiece into the vortex chamber forms a vortex around the axis and passes out of the chamber via the outlet in an axial direction, thereby producing a sound;
a mobile electronic device capable of detecting the sound;
a sound analysis module configured to determine a frequency of the sound; and
a processor configured to determine the respiratory flow rate from said frequency.

3. The apparatus according to claim 2, wherein the flow dependent sound producing device further comprises means for reducing the amplitude of the produced sound comprising a bypass air flow path allowing some of the exhaled air flowing through the mouthpiece to leave the flow dependent sound producing device without passing through the outlet of the vortex chamber.

4. (canceled)

5. The apparatus according to claim 43, wherein the bypass air flow path is arranged to produce a secondary sound having a substantially constant frequency.

6. The apparatus according to claim 5, wherein the secondary sound has a frequency in the ultrasonic range of 20-100 kHz, 30-100 kHz, 40-100 kHz or 50-100 kHz.

7. The apparatus according to claim 4, wherein the bypass air flow path is arranged such that the air passing along the bypass air flow path does not pass into the vortex chamber.

8. The apparatus according to claim 4, wherein the bypass air flow path extends externally around the vortex chamber.

9. The apparatus according to claim 4, wherein the bypass air flow path directs some of the exhaled air to pass out of the device in a straight-through direction via one or more bypass outlets.

10. The method according to claim 1, wherein the analysis to determine a frequency of the sound is carried out in real time.

11. The method according to claim 1, wherein only the determined respiratory flow rate is stored by the electronic device.

12. The apparatus according to claim 2, wherein the mobile electronic device includes a software application available for providing a user interface and displaying respiratory flow rate information to a user.

13. The apparatus according to claim 12, wherein the software application is arranged to display one or more past measurements of respiratory flow rate in addition to a current measurement of respiratory flow rate.

14-15. (canceled)

16. The method according to claim 1, wherein the sound analysis step or sound analysis module is includes calibrated calibration before providing a user with a flow dependent sound producing device.

17. The method according to claim 16, wherein said calibration comprises a test process wherein the relationship between the respiratory flow rate and the determined frequency is verified using a respiratory flow rate meter or spirometer that is different to the flow-dependent sound producing device.

18. (canceled)

19. The method according to claim 1, wherein said frequency is used to determine one or more further parameters relating to the respiratory flow rate chosen from one or more of: Forced Vital Capacity (FVC); Forced Expiratory Volume in 1 Second (FEV1); and the ratio FEV1/FVC.

20. The apparatus according to claim 2, wherein the flow dependent sound producing device comprises a conduit, extending between the mouthpiece and the vortex chamber, arranged to impart at least a tangential component to air flow entering the vortex chamber.

21. The apparatus according to claim 20, wherein the conduit defines a longitudinal axis extending substantially perpendicular to the axis of the vortex chamber or wherein the conduit defines a longitudinal axis extending substantially parallel to the axis of the vortex chamber.

22. (canceled)

23. The apparatus according to claim 2, wherein the outlet comprises an axially extending tube, and the diameter of the tube is smaller than the diameter of the vortex chamber.

24-25. (canceled)

26. A flow dependent sound producing device comprising:
a mouthpiece, for the intake of exhaled air, which is connected to a vortex chamber having an axis and an outlet such that, in use, exhaled air flowing through the mouthpiece into the vortex chamber forms a vortex around the axis and then passes out of the chamber via the outlet in an axial direction, thereby producing a sound, the flow dependent sound producing device further comprising means for reducing the amplitude of the produced sound.

27-41. (canceled)

42. The apparatus according to claim 2, wherein the flow dependent sound producing device has a linear relationship between the volume air flow rate and the frequency of the sound produced.
